(19) United States
(12) Patent Application Publication
(10) Pub. No.: US 2007/0288081 A1
(43) Pub. Date: Dec. 13, 2007

(54) STENT DELIVERY SYSTEM HAVING A STENT STOPPER

(75) Inventors: Randolf Von Oepen, Los Altos Hills, CA (US); Arik Zucker, Zurich (CH); Travis R. Yribarren, San Mateo, CA (US); Thomas Rieth, Hirrlingen (DE); Lorcan Coffey, Tubingen (DE)

Correspondence Address: BEYER WEAVER LLP/Abbott Vascular Devices, P.O. BOX 70250, OAKLAND, CA, 94612-0250, US

(73) Assignee: ABBOTT LABORATORIES, Abbott Park, IL (US)

(21) Appl. No.: 11/747,145

(22) Filed: May 10, 2007

Related U.S. Application Data
(60) Provisional application No. 60/802,046, filed on May 18, 2006.

(51) Int. Cl. A61F 2/06

(52) U.S. Cl. 623/1.11

(57) ABSTRACT

Disclosed are stent delivery apparatus and methods for moving a balloon catheter carrying a stent through a body vessel without the stent slipping from the balloon are provided. In general, the stent delivery system includes a stent stopper that impedes the stent from slipping from the balloon. The stent stopper is sized to serve as a barrier to the stent as it is carried on the balloon. In a specific embodiment, the stent stopper is formed from a material that encircles the proximal end of the balloon and abuts the proximal end of the stent. The stent stopper material expands with expansion of the balloon and collapses with deflation of the balloon. In some embodiments, the stent stopper material has a thickness that is about equal to or greater than the thickness of the stent so as to form a barrier against the stent and impede the stent from slipping off the proximal end of the balloon.

### CROSS REFERENCE TO RELATED PATENT APPLICATION

This application claims priority of co-pending U.S. Provisional Patent Application No. 60/802,046, entitled STENT DELIVERY SYSTEM HAVING A STENT STOPPER, by Randolf Von Oepen et al., filed 18 May 2006, under 35 U.S.C. 119(e), and which application is incorporated herein by reference in its entirety for all purposes.

### BACKGROUND OF THE INVENTION

The present invention relates generally to catheters. More particularly, the present invention relates to stent delivery systems for positioning a stent in a body vessel.

A type of endoprosthesis device, commonly referred to as a stent, may be placed or implanted within a vein, artery or other tubular body organ for treating occlusions, stenoses, or aneurysms of a vessel by reinforcing the wall of the vessel or by expanding the vessel. Stents have been used to treat dissections in blood vessel walls caused by balloon angioplasty of the coronary arteries as well as peripheral arteries and to improve angioplasty results by preventing elastic recoil and remodeling of the vessel wall. Two randomized multicenter trials have recently shown a lower restenosis rate in stent treated coronary arteries compared with balloon angioplasty alone (Serruys, P W et al. New England Journal of Medicine 331: 489-495, 1994, Fischman, D L et al. New England Journal of Medicine 331:496-501, 1994). Stents have been successfully implanted in the urinary tract, the bile duct, the esophagus and the tracheo-bronchial tree to reinforce those body organs, as well as implanted into the neurovascular, peripheral vascular, coronary, cardiac, and renal systems, among others. The term “stent” as used in this Application is a device that is intraluminally implanted within body vessels to reinforce collapsing, dissected, partially occluded, weakened, diseased or abnormally dilated or small segments of a vessel wall.

One common procedure for intraluminally implanting a stent is to use a stent delivery system. A stent is crimped onto a deflated balloon carried on the stent delivery system. The stent delivery system is moved using a guide wire to a target area of a specific body vessel. The balloon is then inflated so as to expand and push the stent against the target area of the vessel. The stent is generally positioned so as to bridge the treated portion of the vessel in order to prevent elastic recoil and restenosis of that segment.

Unfortunately, several factors may cause the stent to slip off the balloon, for example, prior to positioning the stent at the desired target area. In one situation, when the stent delivery system is being guided through a relatively narrow vessel, the stent may be removed from the balloon by contact with the vessel wall. FIG. 1A is a diagrammatic side view, in cross section, of a stent delivery system 104 that is being guided in direction 112 through a narrow body vessel 102. A balloon 114 is carried on the distal end 106a of a flexible tubular structure 106, and a stent 110 is crimped onto the balloon 114 of the stent delivery system 104. The vessel 102 is narrow relative to the balloon and stent of the stent delivery system so that it is possible for the stent to be brushed against the wall of the body vessel while the stent delivery system is guided through the vessel. If the stent 110 is moved against a vessel wall, e.g., wall 115, the stent 110 may slip off the balloon 114 in direction 118. Slip may also occur if the stent 110 is moved against any obstruction such as atheroma or a second stent.

One technique for minimizing this stent slippage is to embed the stent onto the balloon so that portions of the balloon are pushed between the rings (e.g., balloon portions 120c, 120d, and 120e) of the stent and pillow at the distal and proximal ends of the balloon (120a and 120b). However, some stent configurations do not have large enough gaps into which the balloon may be pushed and, accordingly, the balloon is only held in place by the proximal and distal pillowed balloon portions 120a and 120b. Unfortunately, since these pillowed portions 120 are typically sloped, the stent can easily slide up the side of a pillowed balloon portion, e.g., 120a, and off of the balloon.

In some applications the targeted region of a vessel may be at a location where the vessel bifurcates, and this usage contributes to stent slippage in a different manner than a narrow body vessel environment. FIG. 1B is a diagrammatic representation of a side view, in cross-section, of a bifurcated vessel which a stent delivery system 162 is positioned within. As shown, the stent delivery system 162 is positioned in a main branch 152 of the bifurcated vessel. The stent delivery system 162 includes a balloon 164 onto which a stent 156 is crimped. In some procedures, it is desirable to insert a guide wire 151 through the stent into a side branch 154 via ostium 158 prior to placement of the stent 156. In this procedure, the stent is typically crimped less in a proximal area 156b to allow easier insertion of the guide wire through such stent area 156b and into the side vessel 154.

When the guide wire 151 is pushed through the stent and to the side branch 154, the guide wire will tend to apply a force to the stent in direction 165 when pushed against the carina 166. For instance, the stent delivery system may be pushed in direction 168 and exert force on carina 166, which causes an opposite force in direction 165 that results in the stent 156 slipping off the balloon 164 in direction 165.

Accordingly, improved mechanisms for deploying a stent in the vicinity of a body vessel while minimizing stent slippage are needed.

### SUMMARY OF THE INVENTION

Accordingly, stent delivery apparatus and methods for moving a balloon catheter carrying a stent through a body vessel without the stent slipping from the balloon are provided. In general, the stent delivery system includes a stent stopper that impedes the stent from slipping from the balloon. The stent stopper is sized to serve as a barrier to the stent as it is carried on the balloon. In a specific embodiment, the stent stopper is formed from a material that encircles the proximal end of the balloon and abuts the proximal end of the stent. The stent stopper material expands with expansion of the balloon and collapses with deflation of the balloon. In some embodiments, the stent stopper material has a thickness that is about equal to or greater than the thickness of the stent so as to form a barrier against the stent and impede the stent from slipping off the proximal end of the balloon.

In one embodiment, a stent delivery system for inserting a stent in a body vessel is disclosed. The stent delivery system includes an elongate flexible tubular member having an inflation lumen and an inflatable structure carried by a distal portion of the flexible tubular member. The first inflatable structure is in fluid communication with the inflation lumen. The stent delivery system further includes a stent carried by the inflatable structure and a stent stopper carried by a portion of the inflatable structure and adjacent to a first end of the stent. The stent stopper has a thickness that prevents the stent from slipping from the inflatable structure.

In a specific implementation, at least a portion of the stent stopper encircles a proximal end of the inflatable structure and is flexible so as to expand passively with the inflatable structure when the inflatable structure is inflated by the inflation lumen and collapse with the inflatable structure when the inflatable structure is deflated. In a further aspect, the stent stopper has a spiral, tubular shape. In another aspect, the stent stopper has a tubular shape having slots to thereby form a plurality of expandable arms so as to form a tubular stopper portion from which the arms extend. In this aspect, at least a portion of the tubular portion is adhered to the elongate flexible tubular member or inflatable structure and the arms are adjacent to the stent. In a further feature, at least one of the arms of the stent stopper has a thick end portion that is sized to form a barrier to the stent. In yet a further implementation, the thick end portion of the at least one arm has a thickness that is greater than remaining portions of the at least one arm. In another aspect, the stent stopper is a braided tube having a plurality of elongate structures braided together, and at least some ends of the elongate structures are not coupled to each other and are adjacent to the stent to thereby form a barrier to the stent.

In one aspect, the stent stopper is formed from a material selected from a group consisting of a Nitinol material, a stainless steel material, a shape memory alloy, an elastomeric material, and an expandable plastic material. In another aspect, the stent stopper is adhered to the elongate flexible tubular member and/or the inflatable member. In another embodiment, the stent stopper has at least one rounded edge so as to prevent damage to the body vessel and to improve tracking of the stent delivery system.

In another aspect, the invention pertains to a method for inserting a stent into a body vessel using a stent delivery system as described above in any of the stent delivery system embodiments. In general, the stent delivery system is positioned in a body vessel such that the stent is positioned along a target area of the body vessel. The inflatable structure is inflated to thereby expand the stent against the target area of the body vessel and passively expand a portion of the stent stopper that is carried by the inflatable structure. The inflatable structure is also deflated so that the stent stopper returns substantially to its collapsed shape and removing the stent delivery system from the body vessel.

These and other features and advantages of the present invention will be presented in more detail in the following specification of the invention and the accompanying figures which illustrate by way of example the principles of the invention.

### BRIEF DESCRIPTION OF THE DRAWINGS

FIG. 1A is a diagrammatic side view, in cross section, of a stent delivery system.

FIG. 1B is a diagrammatic representation of a side view, in cross-section, of a bifurcated vessel in which a stent delivery system is positioned.

FIG. 2 is a diagrammatic perspective view of a stent delivery system in accordance with a first embodiment of the present invention.

FIG. 3A is a diagrammatic representation, in perspective view, of the stent stopper of FIG. 2 in its collapsed or pre-expanded state.

FIG. 3B shows a portion of the stent stopper of FIGS. 2 and 3A in its expanded state.

FIG. 4 illustrates an alternative stent stopper in accordance with a second embodiment of the present invention.

FIG. 5A is a cross section along line A-A of FIG. 4 illustrating optional thickened arm portions of the stent stopper of FIG. 4.

FIG. 5B is a diagrammatic representation, in perspective view, of the stent stopper of FIG. 4 in its collapsed or pre-expanded state.

FIG. 5C shows a portion of the stent stopper of FIGS. 4 and 5B in its expanded state.

FIGS. 6A and 6B illustrate a stent stopper formed from braided strands in accordance with a third embodiment of the present invention.

### DETAILED DESCRIPTION OF THE SPECIFIC EMBODIMENTS

Reference will now be made in detail to a specific embodiment of the invention. An example of this embodiment is illustrated in the accompanying drawings. While the invention will be described in conjunction with this specific embodiment, it will be understood that it is not intended to limit the invention to one embodiment. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail in order not to unnecessarily obscure the present invention.

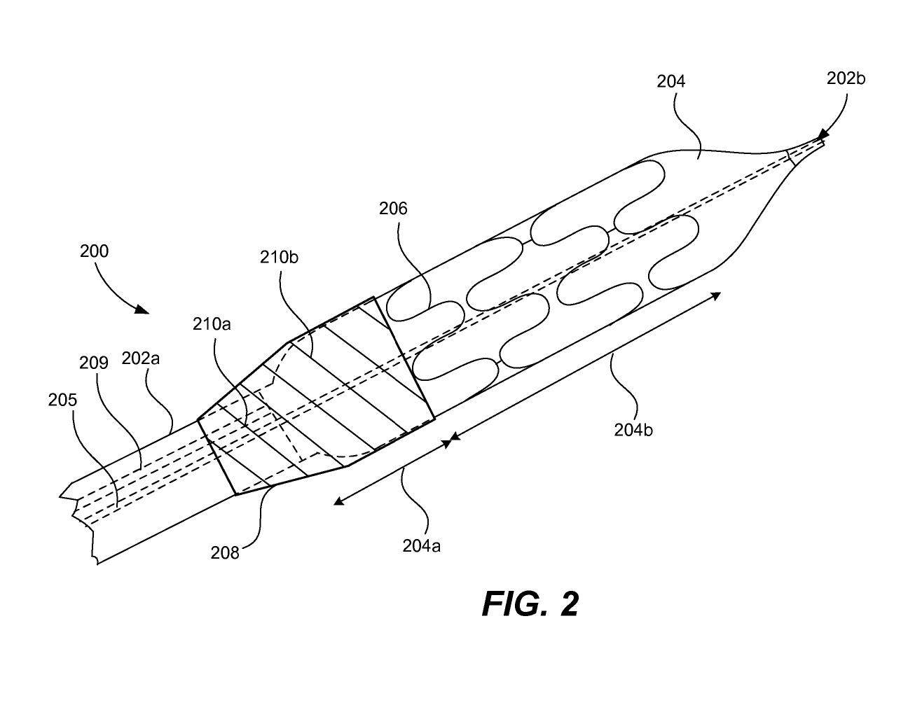

FIG. 2 is a diagrammatic perspective view of a stent delivery system 200 that will be used to introduce some of the concepts that are included in various embodiments of the present invention. The stent delivery system 200 has an elongated flexible tubular member, e.g., tubular member portions 202a and 202b, that is sized suitably for insertion in a vessel of interest. As will be appreciated by those familiar with the art, only the distal, working end of the stent delivery system 200 is shown in this and other figures herein. The length and size of the stent delivery system 200 will typically depend on its desired application and the proximal end of the stent delivery system would typically be outfitted with a suitable handle and ports, valves and other structures for controlling the working (distal) end of the stent delivery system.

In the described embodiment, the stent delivery system is designed for deployment in vascular vessels including coronary vessels. However, in other embodiments, the stent delivery system may be designed for insertion in any body vessel or tubular structure of the body. The flexible tubular member 202 may include any suitable number of lumens. In the illustrated embodiment, the lumens include guide wire lumen 205 and inflation (e.g., fluid supply) lumen 209 although any number of guide wire and/or inflation lumen may be utilized for various applications.

An inflatable structure 204 is mounted near the distal end of the stent delivery system. In the illustrated embodiment, the inflatable structure takes the form of a balloon. It should be appreciated that in many medical applications (e.g., most stent delivery and angioplasty applications) it is generally desirable to provide an inflatable structure that has relatively uniform expansion in all directions. However, various sheaths and other arrangements can be used to encourage the balloon to adopt a particular profile.

Any suitable arrangement may be utilized so that the inflatable balloon is in fluid communication with one or more fluid supply lumen. For instance, the fluid supply lumen 209 may open into the balloon 204 to facilitate inflation of the balloon. The guide wire lumen 205 of the flexible tubular member 202 extends beyond the end of the fluid supply lumens 209 and may support the balloon 204. In this embodiment, the balloon may be attached to the flexible tubular member/guide wire lumen at any appropriate position, as for example along the length of the balloon, at its distal and proximal ends or the like. The balloon may be attached by any suitable mechanism, as for example, by an adhesive, welding, ultrasonic welding, rotation welding, RF energy, laser welding, white light welding, or mechanical bonding.

In some embodiments, the first guide wire lumen 205 ends distal to the distal end of the inflatable member. In other embodiments the guide wire lumen may end exactly at the distal end of the inflatable member. In still other embodiments, the first guide wire lumen will end proximal to the distal end of the inflatable member and the inflatable member (or a structure carried by the inflatable member) therefore forms the distal end of the stent delivery system.

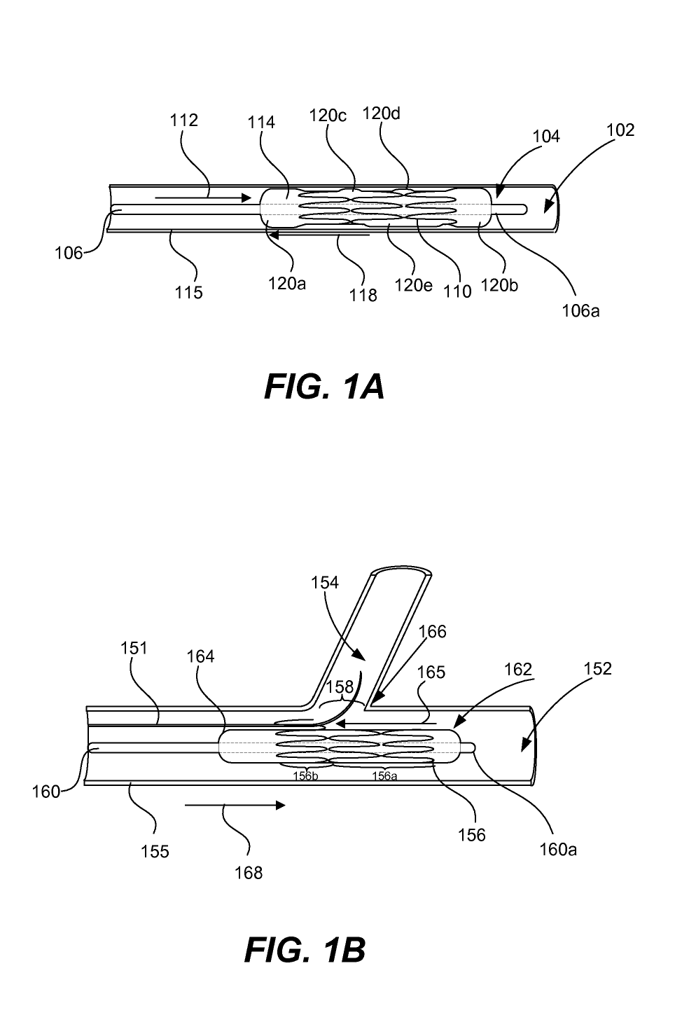

The balloon also carries a stent 206. As shown, distal balloon portion 204b carries stent 206. The stent can be formed from any suitable material and shape that expands with the balloon. The stent may be used as a drug eluting stent to deliver drugs to a vascular region under treatment or to stent a target area of a body vessel, such as an artery. Inflation of the balloon and stent may also serve to both deploy the stent and perform an angioplasty operation. At least a portion of the stent is crimped onto the balloon and expanded upon inflation of such balloon. A portion of the stent may remain uncrimped, or be crimped over a spacing member, to allow a space for an additional guide wire to be inserted, for example, into a side branch as illustrated in FIG. 1B.

In embodiments of the present invention, the stent delivery system also includes a stent stopper carried on a portion of the balloon and adjacent to at least one end of the stent. This stent stopper has a thickness that prevents the stent to slip from the balloon. The stent stopper may have any suitable shape to prevent the stent from slipping from the balloon. In one aspect, the stent stopper encircles a proximal end of the balloon and has a shape that is designed to expand passively with inflation of the balloon and collapse with deflation of the balloon. In its collapsed state, the stopper rests against the balloon and, accordingly, has about the same diameter as the collapsed balloon plus the thickness of the stent stopper wall. The stent stopper is sized and/or shaped so as to form a barrier to the stent, while also minimizing damage to the body vessel wall.

The stent stopper may be fabricated using various shaping technologies, such as laser cutting, injection molding, or fiber winding. Various tools, such as drills and EDM (electrical discharge machining), may be used to cut through or thin the wall of a material so as to form a particular stopper profile or shape. The stent stopper may be formed from any flexible material that passively expands and readily returns substantially to its original shape, such as a Nitinol material, a stainless steel material, a shape memory alloy, an elastomeric material, or an expandable plastic material.

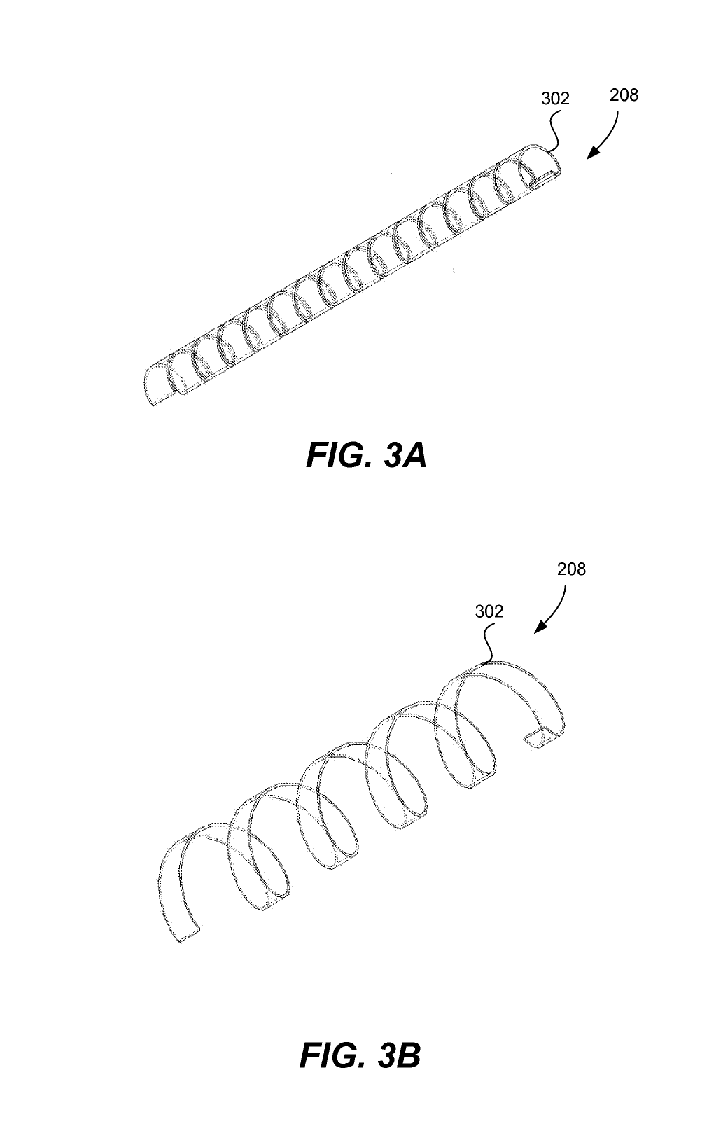

FIG. 2 illustrates merely one embodiment of a stent stopper 208. As shown, the stent stopper 208 has a spiral, tubular shape that is carried by the proximal end of the balloon 204a, as well as a portion of the tubular member 202. The stopper 208 is also adjacent to a proximal end of the stent 206. This spiral stopper may be formed by cutting a tube into a spiral pattern. As shown, the tubular stent stopper 208 has a plurality of cuts, e.g., 210a and 210b, in a spiral pattern. FIG. 3A is a diagrammatic representation, in perspective view, of the stent stopper 208 of FIG. 2 in its collapsed or pre-expanded state. FIG. 3B shows a portion of the stent stopper 208 of FIGS. 2 and 3A in its expanded state. In one aspect, at least the distal end of the spiral material has a thickness (edge 302) that is equal to or greater than the stent thickness so as to prevent the stent from slipping from the balloon. This edge 302 may also be rounded to prevent vessel damage and improve tracking of the stent delivery system.

Additionally, when the balloon is deflated, the stent stopper may help re-fold or deflate the balloon to its pre-inflation diameter by readily returning to its pre-expanded shape. All the stopper embodiments shown herein may have this same feature to help refolding of the balloon. Additionally, all stopper embodiments may have one or more rounded edges (such as adjacent to the stent) to prevent damage to the body vessel when the stent delivery system is being moved through the vessel. Any portion of the stopper may be adhered to the flexible tubular member 202 and/or the balloon 204 using any suitable mechanism, such as by an adhesive bonding, welding, ultrasonic welding, rotation welding, RF energy, laser welding, white light welding, mechanical bonding, heat shrinking, and/or heat staking.

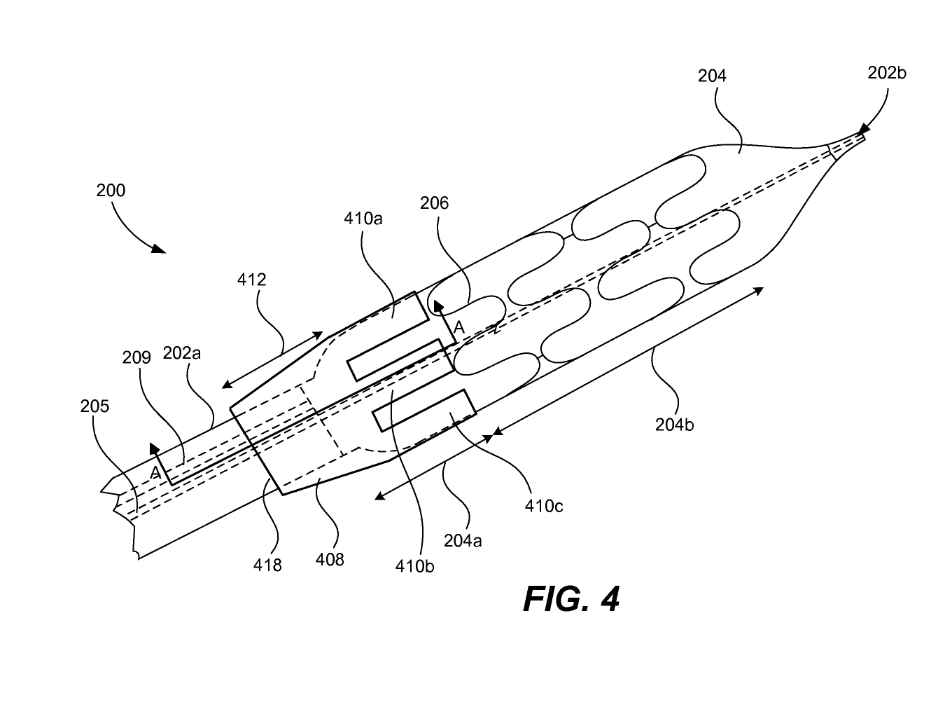

FIG. 4 illustrates an alternative stent stopper 408 in accordance with a second embodiment of the present invention. The stopper 408 is shown as carried by the stent delivery system 200, which has the same components as the stent delivery system shown in FIG. 2. The stopper 408 includes a tubular portion 412 and a plurality of arms, e.g., arms 410a-410c, extending from the tubular portion 412. The stopper 408 may be fabricated by cutting away slots in one end of a tubular shaped material.

Any portion of the tubular portion 412 may be adhered to the balloon or the flexible tubular member 202. In one embodiment, the end 418 of the tubular portion 412 that is opposite the arms is adhered to the flexible tubular member 202. This end 418 may alternatively be adhered to the balloon 204. The arms 410 of the stopper remain unattached from the balloon/tubular member so as to not inhibit the balloon from unfolding. Of course, the stopper arms 410 may also be adhered to the balloon 204 when an unfolded balloon is used, for example. An unfolded balloon may be formed from an elastomeric material that expands substantially in the radial direction when inflated.

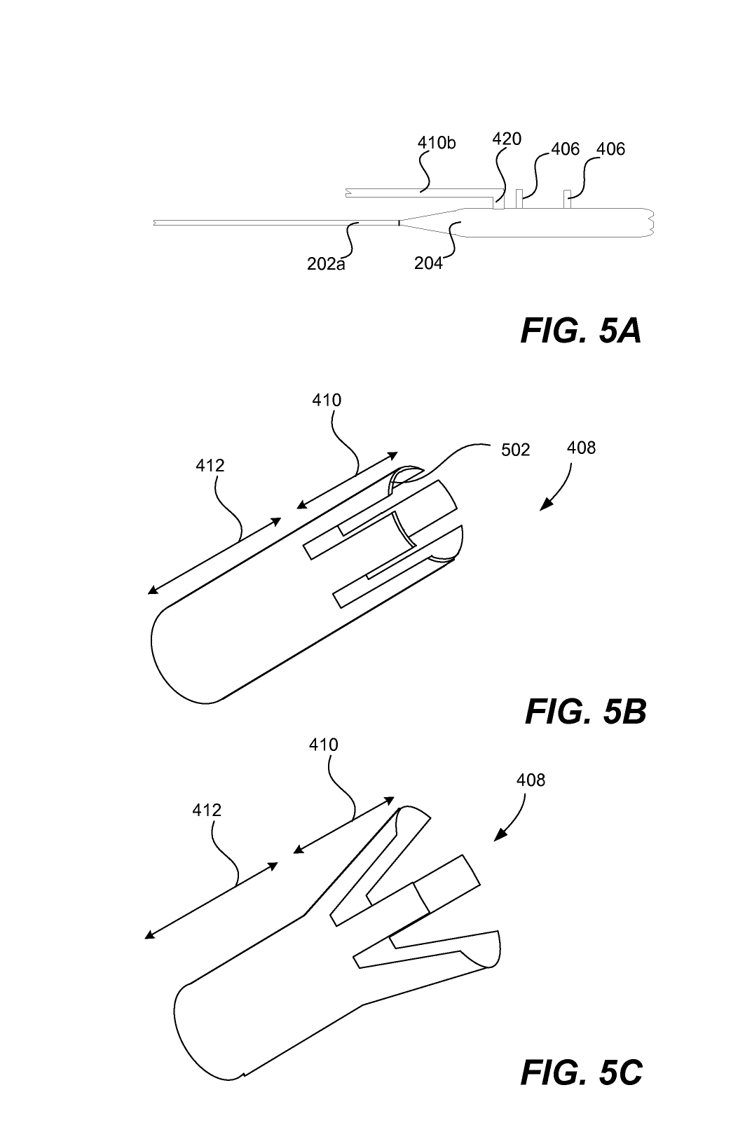

Optionally, each arm may include a thick portion to serve as a more substantial barrier to inhibit stent slippage. That is, each arm's end portion has a greater thickness than the remaining portion of the arm. FIG. 5A is a cross section along line A-A of FIG. 4 illustrating optional thickened arm portions of the stent stopper of FIG. 4. As shown, arm 410b has a thick portion 420 which rests against balloon 204. This thick arm portion 420 is sized so as to substantially inhibit the stent 406 from slipping from the balloon 204. In one implementation, the thick arm portion 420 has a same or greater thickness than the stent thickness (as illustrated).

FIG. 5B is a diagrammatic representation, in perspective view, of the stent stopper 408 of FIG. 4 in its collapsed or pre-expanded state. FIG. 5C shows a portion of the stent stopper 408 of FIGS. 4 and 5B in its expanded state. As shown, the arms 410 of the stopper expand out from the tubular portion 412. At least the distal end 502 of the stopper arms 410 may have a thickness that is equal to or greater than the stent thickness so as to substantially prevent the stent from slipping from the balloon. In this illustration, the arms have a uniform thickness 502 although each arm may have a thicker end portion as shown in FIG. 5A.

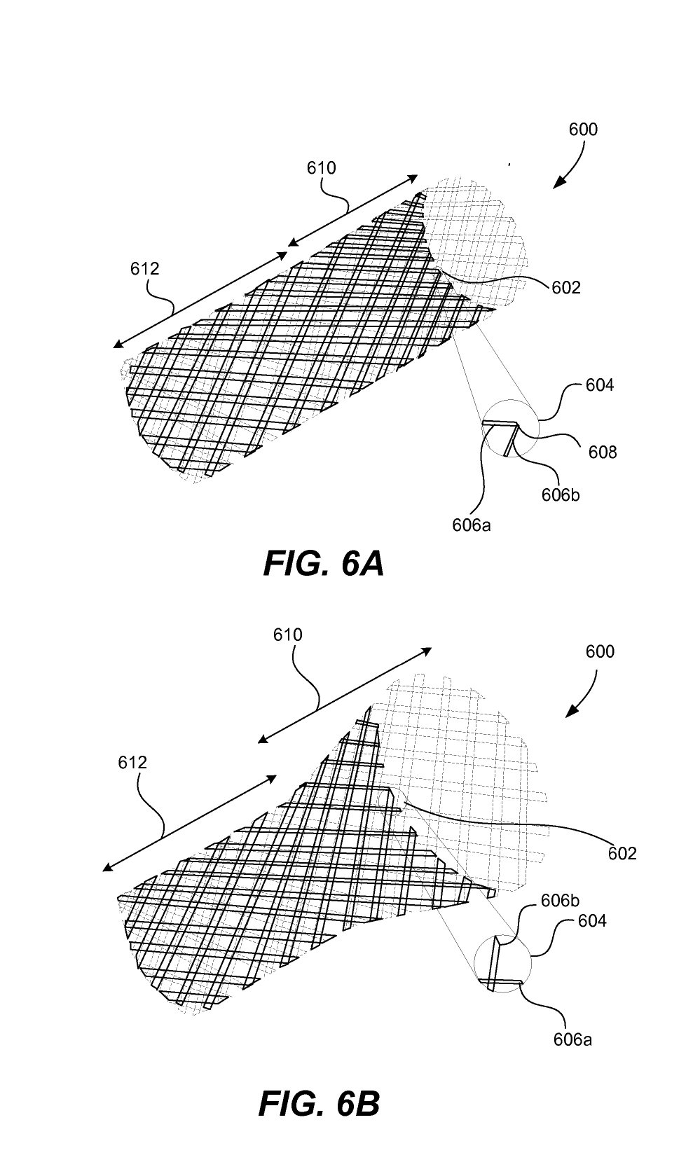

FIGS. 6A and 6B illustrate a stent stopper 600 formed from braided strands in accordance with a third embodiment of the present invention. This stopper 600 may be formed from a plurality of any elongate structures, such as strands, that are braided together. At least some of the strand ends are left uncoupled to each other so as to form an expandable stopper portion 610 that expands with the balloon as shown in FIG. 6B. The remaining stopper portion 612 that is not carried by the balloon remains unexpanded. Of course, the entire stopper may be carried by the balloon such that the entire stopper is expanded along with inflation of the balloon.

View 604 is a magnified view of area 602 having a first strand 606a and a second strand 606b. These strands 606 are not coupled together so that when the expandable portion 610 of the stopper is expanded, the strands 606 spread apart as shown in the view 604 of FIG. 6B. The ends of these uncoupled and expandable strands (possibly along with other coupled-together strands) are positioned adjacent to the stent and serve as a barrier to stent slippage.

Although the strands are described as being not coupled in this embodiment, it is also possible for them to be coupled. This would result in a stent stopper that shortens significantly during expansion, which is not preferred.

A brief technique for using the stent delivery system embodiments of the present invention will now be described. Initially, the stent delivery system is positioned in a body vessel such that the stent is positioned along a target area of the body vessel. During this positioning phase, the stent stopper substantially prevents the stent from slipping from the inflatable structure. The inflatable structure is then inflated to thereby expand the stent against the target area of the body vessel and passively expand a portion of the stent stopper that is carried by the inflatable structure so that the stent stopper may still substantially prevent stent slippage. The inflatable structure is then deflated so that the stent stopper returns substantially to its collapsed shape and removing the stent delivery system from the body vessel. The stent stopper may facilitate the deflation of the inflatable structure.

It should be appreciated that the described stent delivery system arrangement can be useful in a wide variety of interventional procedures. For example, it may be useful in applying stents to one or both branches of a vessel bifurcation with minimal stent slippage. Alternatively, the described arrangements may be useful in facilitating appropriate diagnostic or treatment procedures in a branch of a bifurcation, either together with or separate from a procedure that might be performed in the main branch. For example, a portion of the stent can be crimped less to allow insertion of a guide wire through such uncrimped portion into the side branch, while the stent stopper substantially prevents stent slippage. The procedures may include such procedures as angioplasty procedures, atherectomy procedures, stent delivery procedures, localized drug delivery procedure, visualization procedures, tissue or fluid (e.g., blood) sample acquiring procedures, etc.

Although the foregoing invention has been described in some detail for purposes of clarity of understanding, it will be apparent that certain changes and modifications may be practiced within the scope of the appended claims. For example, the described structure may be incorporated into a simple angioplasty or stent delivery stent delivery system or into different and/or more complicated medical devices. Therefore, the present embodiments are to be considered as illustrative and not restrictive and the invention is not to be limited to the details given herein, but may be modified within the scope and equivalents of the appended claims.

What is claimed is:

1. A stent delivery system for inserting a stent in a body vessel comprising:
an elongate flexible tubular member having an inflation lumen;
an inflatable structure carried by a distal portion of the flexible tubular member, the first inflatable structure being in fluid communication with the inflation lumen;
a stent carried by the inflatable structure; and
a stent stopper carried by a portion of the inflatable structure and adjacent to a first end of the stent, wherein the stent stopper has a thickness that prevents the stent from slipping from the inflatable structure.

2. A stent delivery system as recited in claim 1, wherein at least a portion of the stent stopper encircles a proximal end of the inflatable structure and is flexible so as to expand passively with the inflatable structure when the inflatable structure is inflated by the inflation lumen and collapse with the inflatable structure when the inflatable structure is deflated.

3. A stent delivery system as recited in claim 1, wherein the stent stopper is formed from a material selected from a group consisting of a Nitinol material, a stainless steel material, a shape memory alloy, an elastomeric material, and an expandable plastic material.

4. A stent delivery system as recited in claim 1, wherein the stent stopper is adhered to the elongate flexible tubular member and/or the inflatable member.

5. A stent delivery system as recited in claim 2, wherein the stent stopper has a spiral, tubular shape.

6. A stent delivery system as recited in claim 2, wherein the stent stopper has a tubular shape having slots to thereby form a plurality of expandable arms so as to form a tubular stopper portion from which the arms extend, and wherein at least a portion of the tubular portion is adhered to the elongate flexible tubular member or inflatable structure and the arms are adjacent to the stent.

7. A stent delivery system as recited in claim 6, wherein at least one of the arms of the stent stopper has a thick end portion that is sized to form a barrier to the stent.

8. A stent delivery system as recited in claim 7, wherein the thick end portion of the at least one arm has a thickness that is greater than remaining portions of the at least one arm.

9. A stent delivery system as recited in claim 2, wherein the stent stopper is a braided tube having a plurality of elongate structures braided together.

10. A stent delivery system as recited in claim 9, wherein at least some ends of the elongate structures are not coupled to each other and are adjacent to the stent to thereby form a barrier to the stent.

11. A stent delivery system as recited in claim 1, wherein the stent stopper has at least one rounded edge so as to prevent damage to the body vessel.

12. A method for inserting a stent into a body vessel using a stent delivery system, wherein the stent delivery system comprises an elongate flexible tubular member having an inflation lumen, an inflatable structure carried by a distal portion of the flexible tubular member, the first inflatable structure being in fluid communication with the inflation lumen, a stent carried by the inflatable structure, and a stent stopper carried by a portion of the inflatable structure and adjacent to a first end of the stent, wherein the stent stopper has a thickness that prevents the stent from slipping from the inflatable structure, the method comprising:
positioning the stent delivery system in a body vessel such that the stent is positioned along a target area of the body vessel;
inflating the inflatable structure to thereby expand the stent against the target area of the body vessel and passively expand a portion of the stent stopper that is carried by the inflatable structure; and
deflating the inflatable structure so that the stent stopper returns substantially to its collapsed shape and removing the stent delivery system from the body vessel.
